(19) United States
(12) Patent Application Publication
(10) Pub. No.: US 2025/0115897 A1
(43) Pub. Date: Apr. 10, 2025

(54) IN VITRO TRANSCRIPTION DNA PURIFICATION AND RECYCLING

(71) Applicant: ModernaTX, Inc., Cambridge, MA (US)

(72) Inventors: Amy E. Rabideau, Cambridge, MA (US); Ivan Manzano, Cambridge, MA (US); Marcus Duvall, Cambridge, MA (US)

(73) Assignee: ModernaTX, Inc., Cambridge, MA (US)

(21) Appl. No.: 18/728,664

(22) PCT Filed: Jan. 13, 2023
(86) PCT No.: PCT/US2023/010757

§ 371 Date: Jul. 12, 2024

Related U.S. Application Data
(60) Provisional application No. 63/299,847, filed on Jan. 14, 2022.

(51) Int. Cl. C12N 15/10; C12P 19/34

(52) U.S. Cl. CPC C12N 15/101; C12P 19/34

(57) ABSTRACT

Provided herein are methods of preparing recycled template DNA solution from in vitro transcription (IVT) reaction product comprising template DNA. In some embodiments. the methods comprise removing mRNA from an IVT reaction product and removing one or more additional IVT reaction components and/or impurities to obtain a recycled template DNA solution. Also provided herein are systems for in vitro transcription (IVT) for producing mRNA.

### RELATED APPLICATIONS

This application claims priority under 35 U.S.C. § 119 (c) of U.S. Provisional Application, U.S. Ser. No. 63/299,847, filed Jan. 14, 2022, which is incorporated herein by reference.

### BACKGROUND

Messenger RNA (mRNA) is an emerging alternative to conventional small molecule and protein therapeutics due to the potency and programmability of mRNA. mRNA encoding a desired therapeutic protein can be administered to a subject for in vivo expression of the protein to therapeutic effect, such as vaccination or replacement of a protein encoded by a mutated gene. In vitro transcription of a DNA template using an RNA polymerase is a useful method of producing mRNAs for therapeutic applications. However, after use, the DNA template is discarded with other IVT reaction residual products and for each subsequent in vitro transcription reaction, a new DNA template is used.

### SUMMARY

Provided herein are methods of preparing a recycled template DNA solution, from an in vitro transcription reaction. In vitro transcription, in which an RNA polymerase uses a DNA template to produce an RNA transcript, is useful for generating mRNA, but once the DNA template is used once, it is discarded, and a new DNA template must be used each time in vitro transcription occurs.

Accordingly, the present disclosure provides, in some aspects, a method of preparing a recycled template DNA solution, the method comprising: removing mRNA from an in vitro transcription (IVT) reaction product to obtain a solution comprising the template DNA and one or more additional IVT reaction components and/or impurities; and then removing the one or more additional IVT reaction components and/or impurities from the solution to obtain a recycled template DNA solution.

In some embodiments, the one or more additional IVT reaction components comprise a RNA polymerase.

In some embodiments, the one or more impurities comprise tailless mRNA and/or non-binding mRNA.

In some embodiments, the recycled template DNA solution is substantially free of mRNA and RNA polymerase.

In some embodiments, the method further comprises performing an IVT reaction using the recycled template DNA solution.

In some embodiments, the one or more additional IVT reaction components and/or impurities are removed using membrane chromatography.

In some embodiments, the method further comprises performing at least four cycles of the membrane chromatography.

In some embodiments, a high productivity anion exchange chromatography membrane is used in the membrane chromatography.

In some embodiments, the method further comprises purifying the IVT reaction product using tangential flow filtration (TFF) before and/or after removing the mRNA from the IVT reaction product.

In some embodiments, the performed IVT reaction comprises combining the recycled template DNA with a RNA polymerase selected from a T7, T3 or SP6 RNA polymerase.

In some embodiments, the RNA polymerase is a T7 variant RNA polymerase.

In some embodiments, the recycled template DNA is plasmid DNA (pDNA), PCR-amplified DNA, or complementary DNA (cDNA).

In some embodiments, the recycled template DNA is 2-10 kilo base pairs (kbp) in length.

In some aspects, the present disclosure provides a system for in vitro transcription (IVT) for producing mRNA, the system, comprising
an IVT reaction chamber comprising IVT reaction components, wherein the IVT reaction components comprise at least a template DNA,
a first system for separating IVT-produced mRNA from a residual reaction product comprising the template DNA and
a second system for separating template DNA from one or more additional IVT reaction components and/or impurities of the residual reaction product.

In some embodiments, the second system performs membrane chromatography.

In some embodiments, the membrane chromatography comprises a high productivity anion exchange chromatography membrane.

In some embodiments, the system further comprises a third system for adding the separated template DNA back to the reaction chamber.

In some aspects, the present disclosure provides a composition comprising, a recycled template DNA and wherein the composition comprises at least one of a reaction compound selected from a tailless RNA, a non-binding RNA, and an RNA polymerase and wherein less than 1% of the composition comprises the reaction compound.

In some embodiments, the recycled template DNA is plasmid DNA (pDNA), PCR-amplified DNA, or complementary DNA (cDNA).

In some embodiments, the recycled template DNA is plasmid DNA (pDNA).

In some embodiments, the recycled template DNA is 2-10 kilo base pairs (kbp) in length.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising.” or “having.” “containing”, “involving”, and variations thereof herein, is meant to encompass the items listed thereafter and equivalents thereof as well as additional items.

### BRIEF DESCRIPTION OF THE DRAWINGS

FIGS. 1A-1B show recycled pDNA NTP consumption (FIG. 1A) and control pDNA NTP consumption (FIG. 1B).

FIGS. 2A-2B show % tail purity and IVT productivity of recycled pDNA (FIG. 2A) and control pDNA (FIG. 2B).

### DETAILED DESCRIPTION

Methods for preparing mRNA can normally be achieved using in vitro transcription (IVT) reactions. The reagents involved in IVT reactions include at least a DNA template, a polymerase, and nucleotides. While IVT reactions are commonly performed there are some limitations in the transcription reaction that can result in inadequate results. For instance, under some conditions the transcript may not be generated or may include errors. The use of a poor-quality DNA template, for example, has been shown to interfere with IVT reactions. Reaction contaminants (such as ethanol or salts which haven't been properly removed from the DNA template or RNase contamination) can also interfere with efficient IVT reactions. Efforts to improve the quality of IVT reaction products typically are focused on efforts to enhance the reaction and/or quality of the reagents used in the reaction.

Surprisingly, it was discovered that template DNA which has been used in an IVT reaction can be recaptured from the reaction residual, adequately processed and reused in a new IVT reaction, without disrupting or causing interference with the new IVT reaction. Thus, recycled template DNA is provided herein, as well as methods of using the recycled template DNA in IVT reactions and related systems.

The present disclosure relates to methods of preparing a recycled template DNA solution from an in vitro transcription (IVT) reaction. Generally, after mRNA is transcribed in an IVT reaction, the mRNA is separated from the remainder of the IVT reaction products and purified for further use. The remainder of the IVT reaction products which are typically discarded following the reaction are referred to as an IVT residual reaction product. The IVT reaction residual product includes at least a template DNA. The residual product also often includes a RNA polymerase and partially formed RNA or RNA fragments such as tailless RNA, non-binding RNA, etc. This IVT residual reaction product is further processed in order to produce a recycled template DNA which can be used in subsequent (e.g., second, third, fourth, etc.) IVT reactions. Unexpectedly it was found that high quality recycled template DNA could be produced and that it could be used in subsequent IVT reactions to produce high quality mRNA product.

Thus, in some aspects, the methods provided herein comprise performing an IVT reaction, removing mRNA from an IVT residual reaction product to obtain a solution comprising a template DNA and one or more additional IVT reaction components and/or impurities from the solution to obtain a recycled template DNA solution.

The template DNA can serve as a nucleic acid template for RNA polymerase. A template DNA may include a polynucleotide encoding a polypeptide of interest (e.g., an antigenic polypeptide). A template DNA, in some embodiments, includes an RNA polymerase promoter (e.g., a T7 RNA polymerase promoter) located 5′ from and operably linked to polynucleotide encoding a polypeptide of interest. A template DNA may also include a nucleotide sequence encoding a polyadenylation (polyA) region located at the 3′ end of the gene of interest. In some embodiments, a template DNA comprises plasmid DNA (pDNA). As used herein, “plasmid DNA” or “pDNA” refers to an extrachromosomal DNA molecule that is physically separated from chromosomal DNA in a cell and can replicate independently. In some embodiments, plasmid DNA is isolated from a cell (e.g., as a plasmid DNA preparation). In some embodiments, plasmid DNA comprises an origin of replication, which may contain one or more heterologous nucleic acids, that may serve as a template for RNA polymerase. Plasmid DNA may be circularized or linear (e.g., plasmid DNA that has been linearized by a restriction enzyme digest). Typically, the template DNA is synthesized in the form of a plasmid and then used in the first IVT reaction.

A recycled template DNA refers to template DNA that has already been used in an IVT reaction to prepare mRNA. The recycled template DNA is isolated and purified from a first IVT residual reaction product. The sequence and sequence elements of the recycled template DNA are the same as those of the template DNA. In some embodiments, the recycled template DNA is plasmid DNA (pDNA), PCR-amplified DNA, or complementary DNA (cDNA). In some embodiments, the recycled template DNA is plasmid DNA (pDNA). In some embodiments, the recycled template DNA is 2-10 kilo base pairs (kbp) in length. For example, in some embodiments, the recycled template DNA is 2-10 kbp, 4-10 kbp, 6-10 kbp, 8-10 kbp, 2-8 kbp, 4-8 kbp, 6-8 kbp, 2-6 kbp, 4-6 kbp, 2-4 kbp in length. In some embodiments, the recycled template DNA is 2, 3, 4, 5, 6, 7, 8, 9, or 10 kbp in length.

### First IVT Reaction

An in vitro transcription (IVT) is used to produce an RNA transcript (e.g., mRNA transcript). IVT is a process that comprises contacting a DNA template with an RNA polymerase (e.g., a T7 RNA polymerase, a T7 RNA polymerase variant, etc.) under conditions that result in the production of the RNA transcript. IVT conditions typically require a purified DNA template containing a promoter, nucleoside triphosphates, a buffer system that includes dithiothreitol (DTT) and magnesium ions, and an RNA polymerase. The exact conditions used in the transcription reaction depend on the amount of RNA needed for a specific application. Typical IVT reactions are performed by incubating a DNA template with an RNA polymerase and nucleoside triphosphates, including GTP, ATP, CTP, and UTP (or nucleotide analogs) in a transcription buffer. An RNA transcript having a 5′ terminal guanosine triphosphate is produced from this reaction. In the first IVT reaction the template DNA is newly synthesized and purified. It has not been used in a prior IVT reaction.

In some embodiments, an IVT reaction uses an RNA polymerase selected from the group consisting of T7 RNA polymerase, T3 RNA polymerase, K11 RNA polymerase, and SP6 RNA polymerase. In some embodiments, a wild-type T7 polymerase is used in an IVT reaction. In some embodiments, a mutant T7 polymerase is used in an IVT reaction. In some embodiments, a T7 RNA polymerase variant comprises an amino acid sequence that shares at least 50%, 60%, 70%, 80%, 90%, 95%, or 99% identity with a wild-type T7 (WT T7) polymerase. In some embodiments, the T7 polymerase variant is a T7 polymerase variant described by International Application Publication Number WO2019/036682 or WO2020/172239, the entire contents of each of which are incorporated herein by reference.

The nucleoside triphosphates (NTPs) as provided herein may comprise unmodified or modified ATP, modified or unmodified UTP, modified or unmodified GTP, and/or modified or unmodified CTP. In some embodiments, NTPs of an IVT reaction comprise unmodified ATP. In some embodiments, NTPs of an IVT reaction comprise modified ATP. In some embodiments, NTPs of an IVT reaction comprise unmodified UTP. In some embodiments, NTPs of an IVT reaction comprise modified UTP. In some embodiments, NTPs of an IVT reaction comprise unmodified GTP. In some embodiments, NTPs of an IVT reaction comprise modified GTP. In some embodiments, NTPs of an IVT reaction comprise unmodified CTP. In some embodiments, NTPs of an IVT reaction comprise modified CTP.

In some embodiments, an RNA transcript (e.g., mRNA transcript) produced by the IVT reaction includes a modified nucleobase selected from pseudouridine (ψ), 1-methylpseudouridine (m¹ψ), 5-methoxyuridine (mo⁵U), 5-methylcytidine (m⁵C), α-thio-guanosine and α-thio-adenosine. In some embodiments, an RNA transcript (e.g., mRNA transcript) includes a combination of at least two (e.g., 2, 3, 4 or more) of the foregoing modified nucleobases. Thus, in some embodiments, the IVT reaction includes such modified nucleobases.

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 1-methylpseudouridine (m¹ψ), meaning that all uridine residues in the mRNA sequence are replaced with 1-methylpseudouridine (m¹ψ). Similarly, a polynucleotide can be uniformly modified for any type of nucleoside residue present in the sequence by replacement with a modified residue such as any of those set forth above. Alternatively, the polynucleotide (e.g., RNA polynucleotide, such as mRNA polynucleotide) may not be uniformly modified (e.g., partially modified, part of the sequence is modified). Each possibility is achievable using an IVT reaction as disclosed herein. In some embodiments, modified nucleotides are included in an IVT mixture, and are incorporated randomly during transcription, such that the RNA contains a mixture of modified nucleotides and unmodified nucleotides.

The buffer system of an IVT reaction mixture may vary. In some embodiments, the buffer system contains Tris. The concentration of tris used in an IVT reaction, for example, may be at least 10 mM, at least 20 mM, at least 30 mM, at least 40 mM, at least 50 mM, at least 60 mM, at least 70 mM, at least 80 mM, at least 90 mM, at least 100 mM, at least 110 mM, at least 120 mM, at least 150 mM, at least 200 mM, at least 300 mM, at least 400 mM, at least 500 mM, or at least 600 mM tris. In some embodiments, the concentration of tris is 20-60 mM or 10-100 mM.

The buffer system of an IVT reaction mixture may vary. In some embodiments, the buffer system contains phosphate. The concentration of phosphate used in an IVT reaction, for example, may be at least 10 mM, at least 20 mM, at least 30 mM, at least 40 mM, at least 50 mM, at least 60 mM, at least 70 mM, at least 80 mM, at least 90 mM, at least 100 mM, at least 110 mM, at least 120 mM, at least 150 mM, at least 200 mM, at least 300 mM, at least 400 mM, at least 500 mM, or at least 600 mM phosphate. In some embodiments, the concentration of phosphate is 20-60 mM or 10-100 mM phosphate.

The buffer system of an IVT reaction mixture may vary. In some embodiments, the buffer system contains citrate. The concentration of citrate used in an IVT reaction, for example, may be at least 10 mM, at least 20 mM, at least 30 mM, at least 40 mM, at least 50 mM, at least 60 mM, at least 70 mM, at least 80 mM, at least 90 mM, at least 100 mM, at least 110 mM, at least 120 mM, at least 150 mM, at least 200 mM, at least 300 mM, at least 400 mM, at least 500 mM, or at least 600 mM citrate. In some embodiments, the concentration of tris is 20-60 mM or 10-100 mM citrate.

In some embodiments, the buffer system contains dithiothreitol (DTT). The concentration of DTT used in an IVT reaction, for example, may be at least 1 mM, at least 5 mM, at least 50 mM, at least 75 mM, or at least 100 mM DTT. In some embodiments, the concentration of DTT used in an IVT reaction is 1-50 mM or 5-50 mM. In some embodiments, the concentration of DTT used in an IVT reaction is 5 mM.

In some embodiments, the buffer system contains magnesium. In some embodiments, the molar ratio of NTP to magnesium ions (Mg²⁺; e.g., MgCl₂) present in an IVT reaction is 1:1 to 1:10. For example, the molar ratio of NTP to magnesium ions may be 1:0.25, 1:0.5, 1:1, 1:2, 1:3, 1:4. 1:5, 1:6, 1:7, 1:8, 1:9, or 1:10.

In some embodiments, the molar ratio of NTP to magnesium ions (Mg²⁺; e.g., MgCl₂) present in an IVT reaction is 1:1 to 1:5. For example, the molar ratio of NTP to magnesium ions may be 1:1, 1:2, 1:3, 1:4 or 1:5.

In some embodiments, the buffer system contains Tris-HCl, spermidine (e.g., at a concentration of 1-30 mM), TRITON® X-100 (polyethylene glycol p-(1,1,3,3-tetramethylbutyl)-phenyl ether) and/or polyethylene glycol (PEG).

In some embodiments, IVT methods further comprise a step of separating (e.g., purifying) in vitro transcription products (e.g., mRNA) from other reaction components (“IVT residual reaction product”). The IVT reaction is typically performed in a vesicle, referred to herein as a reaction chamber. Once the reaction is complete the reaction components may be transferred, either manually or automatically (e.g. through automated equipment or systems) to a separation chamber, wherein the separation step is performed.

In some embodiments, the separation step performed on the completed IVT reaction product comprises performing a first filtration step on the IVT reaction mixture. The first filtration step may be a procedure such as chromatography. In some embodiments, the method comprises reverse phase chromatography. In some embodiments, the method comprises reverse phase column chromatography. In some embodiments, the chromatography comprises size-based (e.g., length-based) chromatography. In some embodiments, the method comprises size exclusion chromatography. In some embodiments, the chromatography comprises oligo-dT chromatography. Chromatographic methods that achieve higher recoveries and purities, more predictable scalability, and better reproducibility include, for instance, affinity, anion-exchange, hydrogen-bonding, hydrophobic-interaction, and reversed-phase chromatography methods. During the separation step the mRNA is separated from the IVT residual reaction product. For example, in some embodiments, the mRNA is removed from the IVT residual reaction product to obtain a solution comprising a template DNA and one or more additional IVT reaction components (e.g., proteins (e.g., enzymes), RNA polymerase, reaction buffer, nucleoside triphosphates (NTPs)) and/or impurities (e.g., tailless mRNA, mRNA fragments, non-binding mRNA). The mRNA can be further purified and processed for use as a drug product or other purposes such as research.

Some embodiments comprise filtering the IVT reaction product (e.g., using ultrafiltration such as tangential flow filtration) to remove small components, such as nucleotides, salts, and other IVT reagents. Such filtering can be performed before and/or after removing the mRNA from the IVT residual reaction product.

The IVT residual reaction product that the mRNA is removed from is conventionally discarded. Contrary to conventional methods, some aspects of the disclosure comprising collecting IVT residual reaction product (e.g., in a collection chamber) for further processing, e.g., to isolate DNA template. An IVT residual reaction product, as used herein, is a composition that comprises template DNA used in the first IVT reaction, one or more additional IVT reaction components and/or impurities. The one or more additional IVT reaction components and/or impurities include at least an RNA polymerase and optionally, RNA fragments or structures distinct from the mRNA, and free nucleotides and buffer components. The RNA fragments may comprise truncated RNA, double stranded RNA, tailless RNA, non-coding RNA etc.

### Isolation of Recycled DNA After IVT

In some embodiments, the IVT residual reaction product is subjected to an additional separation or isolation step in order to separate the template DNA from the other reaction components. Thus, in the methods provided herein, once the mRNA is separated from the other reaction components, a recycled template DNA solution can be produced from the IVT residual reaction product. For instance, the IVT residual reaction product in the collection chamber can be subjected to a separation step. Such a separation step can separate DNA from other reaction components (e.g., proteins (e.g., enzymes), RNA polymerase, reaction buffer, nucleoside triphosphates (NTPs)) and/or impurities (e.g., tailless mRNA, mRNA fragments, non-binding mRNA). In some embodiments, the methods provided herein comprise removing one or more additional IVT reaction components and/or impurities from the residual reaction product to obtain a recycled template DNA solution.

In some embodiments, the separation step is carried out using column chromatography (e.g., ion-exchange chromatography, affinity chromatography). Chromatographic media may include but is not limited to resin (e.g. DEAE, Poros™ resins) and convective materials such as membranes (e.g., Natrix® membranes) and monoliths (e.g., CIM® (Convective Interaction Media) monoliths). In some embodiments, the separation step is carried out using column chromatography using resin-based columns.

In some embodiments, the separation step is carried out using monolith chromatography. As used herein, “monolith chromatography” is a method of purification that provides highly selective separations through adsorption/binding interactions based on ion-exchange, affinity, reversed-phase, and/or hydrophobic interactions. Monolith chromatography uses a monolith support comprising a single, continuous, highly interconnected, and porous organic or inorganic stationary separation block, with functional ligands immobilized on the surfaces.

In some embodiments, the separation step is carried out using membrane chromatography. As used herein, “membrane chromatography” is a method of purification that provides highly selective separations through adsorption/binding interactions based on ion-exchange, affinity, reversed-phase, and/or hydrophobic interactions. Membrane chromatography uses membranes with micron-size pores that have functional ligands on the inner pore surface throughout the membrane structure.

In some embodiments, a high productivity anion exchange chromatography membrane is used to remove the remainder of the IVT residual reaction product components and/or impurities to obtain a recycled template DNA solution. The membrane may be, for instance, comprised of a porous membrane scaffold. In some embodiments the anion exchange chromatography membrane contains a high density of a ligand such as a quaternary amine ligand for promoting effective capture. The ligands may be directly polymerized within the porous membrane scaffold. Membranes having interconnected pore structure and high ligand density are particularly useful for enabling fast flow rates with high throughput. In some embodiments, such membranes can also maintain high levels of impurity reduction. The membrane, in some embodiments, may be a Natrix® Q chromatography membrane. A Natrix® membrane is a high capacity, high throughput strong anion exchange chromatography membrane designed for single use per batch biomolecule purification, that is commercially available from Millipore Sigma.

In some embodiments, the separation step comprises at least one cycle of separation. However, multiple cycles can be performed in order to maximize the separation of the other residual reactants from the recycled template DNA solution. In some embodiments the separation stem may comprised at least 2 cycles, at least three cycles, at least four, at least five, at least six, at least seven, at least eight, at least 9, or at least 10 cycles) of chromatography or filtration.

In some embodiments, the methods further comprise additional steps of purification of the IVT reaction product in order to produce a pure recycled template DNA solution for use in subsequent IVT reactions. In some embodiments, the additional steps of purification of the IVT reaction product occurs before and/or after (e.g., before, after, or both before and after) removing the mRNA from the IVT reaction product. The additional purification steps may involve any type of purification useful for purifying DNA. For instance, purification can be performed using tangential flow filtration (TFF). In TFF, a mixture flows over a filtration membrane (TFF membrane) comprising pores, with the pores of the membrane being oriented perpendicular to the direction of flow. Components of the mixture flow through the pores, if able, while components that do not pass through the pores are retained in the mixture. TFF thus removes smaller impurities, such as peptide fragments, amino acids, and nucleotides from a mixture, while larger molecules, such as the recycled DNA template, are retained in the mixture.

The size of the pores of the TFF membrane affect which components are filtered (removed) from the mixture and which are retained in the mixture. Generally, TFF membranes are characterized in terms of a molecular weight cutoff, with components smaller than the molecular weight cutoff being removed from the mixture during TFF, while components larger than the molecular weight cutoff being retained in the mixture. In some embodiments, the tangential flow filtration comprises using a TFF membrane with a molecular weight cutoff of 500 kDa or less, 200 kDa or less, or 100 kDa or less. In some embodiments, the tangential flow filtration comprises using a TFF membrane with a molecular weight cutoff of 500 kDa or less. In some embodiments, the tangential flow filtration comprises using a TFF membrane with a molecular weight cutoff of 400 kDa or less. In some embodiments, the tangential flow filtration comprises using a TFF membrane with a molecular weight cutoff of 300 kDa or less. In some embodiments, the tangential flow filtration comprises using a TFF membrane with a molecular weight cutoff of 200 kDa or less. In some embodiments, the tangential flow filtration comprises using a TFF membrane with a molecular weight cutoff of 100 kDa or less.

In some embodiments, the recycled template DNA solution is substantially free of additional IVT reaction components and/or impurities. In some embodiments, the recycled template DNA solution is substantially free of mRNA and RNA polymerase.

Any of the methods provided herein may further comprise performing a second IVT reaction using the recycled template DNA solution in the reaction.

### Second or Subsequent IVT Reaction

Once the recycled template DNA is isolated and further processed it may be used in a second or subsequent IVT reaction. The second or subsequent IVT reaction can be a standard IVT reaction, and/or can be similar to the first IVT reaction, but that some or all of the DNA template in the reaction is recycled template DNA. In some embodiments 100% of the template DNA used in the reaction is recycled template DNA. In other embodiments at least 10%, 20%, 30%, 40%, 50%, 55%, 60%, 65%, 70%, 75%, 80%, 85%, 90%, 95%, 98%, or 99% of the template DNA used in the reaction is recycled template DNA. In some embodiments, the second or subsequent IVT reaction comprises combining the recycled template DNA with a RNA polymerase (e.g., T7, T3, or SP6).

Following the second or subsequent IVT reaction, the recycled template DNA may be isolated, separated from the mRNA and the additional IVT reaction components and/or impurities and purified for use in subsequent IVT reactions.

### Systems for IVT

Some aspects of the present disclosure provide systems for IVT for producing mRNA, such as automated systems. In some embodiments, the systems provide an IVT reaction chamber comprising IVT reaction components, wherein the IVT reaction components comprise at least a template DNA and polymerase, a first system for separating IVT-produced mRNA from an IVT residual reaction product comprising the template DNA, a second system for separating template DNA from one or more additional IVT reaction components and/or impurities of the residual reaction product. In some embodiments the recycled template DNA may be collected from the system, optionally further processed and fed back into the system. For example, in some embodiments, the systems comprise a third system for adding the separated template DNA back to the reaction chamber for a second or subsequent IVT reaction.

### Recycled DNA Compositions

Some aspects of the present disclosure provide compositions comprising recycled DNA produced by any of the methods provided herein. In some embodiments, a composition comprises recycled template DNA, wherein the composition comprises at least one of a reaction compound selected from a tailless RNA, a non-binding RNA, and an RNA polymerase and wherein less than 1% (e.g., less than 1%, less than 0.8%, less than 0.6%, less than 0.4%, or less than 0.2%) of the composition comprises the reaction compound. In some embodiments, the recycled template DNA is plasmid DNA (pDNA), PCR-amplified DNA, or complementary DNA (cDNA). In some embodiments, the recycled template DNA is plasmid DNA (pDNA). In some embodiments, the recycled template DNA is 2-10 kilo base pairs (kbp) in length. For example, in some embodiments, the recycled template DNA is 2-10 kbp, 4-10 kbp, 6-10 kbp, 8-10 kbp, 2-8 kbp, 4-8 kbp, 6-8 kbp, 2-6 kbp, 4-6 kbp, 2-4 kbp in length. In some embodiments, the recycled template DNA is 2, 3, 4, 5, 6, 7, 8, 9, or 10 kbp in length.

### Nucleic Acids

Aspects of the present disclosure relate to compositions and regents comprising nucleic acids and methods of producing nucleic acids using such compositions and reagents. As used herein, the term “nucleic acid” includes multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G))). The term nucleic acid includes polyribonucleotides as well as polydeoxyribonucleotides. The term nucleic acid also includes polynucleosides (i.e., a polynucleotide minus the phosphate) and any other organic base containing polymer. Non-limiting examples of nucleic acids include chromosomes, genomic loci, genes or gene segments that encode polynucleotides or polypeptides, coding sequences, non-coding sequences (e.g., intron, 5′-UTR, or 3′-UTR) of a gene, pri-mRNA, pre-mRNA, cDNA, mRNA, etc. A nucleic acid (e.g., mRNA) may include a substitution and/or modification. In some embodiments, the substitution and/or modification is in one or more bases and/or sugars. For example, in some embodiments a nucleic acid (e.g., mRNA) includes nucleotides having an organic group, such as a methyl group, attached to a nucleic acid base at the N6 position. Thus, in some embodiments, an mRNA includes one or more N6-methyladenosine nucleotides. A phosphate, sugar, or nucleic acid base of a nucleotide may also be substituted for another phosphate, sugar, or nucleic acid base. For example, a uridine base may be substituted for a pseudouridine base, in which the uracil base is attached to the sugar by a carbon-carbon bond rather than a nitrogen-carbon bond. Thus, in some embodiments, a nucleic acid (e.g., mRNA) is heterogeneous in backbone composition thereby containing any possible combination of polymer units linked together such as peptide-nucleic acids (which have an amino acid backbone with nucleic acid bases).

The nucleic acid sequences of the present invention include nucleic acid sequences that have been removed from their naturally occurring environment, recombinant or cloned DNA isolates, and chemically synthesized analogues or analogues biologically synthesized by heterologous systems.

An “engineered nucleic acid” is a nucleic acid that does not occur in nature. It should be understood, however, that while an engineered nucleic acid as a whole is not naturally occurring, it may include nucleotide sequences that occur in nature. In some embodiments, an engineered nucleic acid comprises nucleotide sequences from different organisms (e.g., from different species). For example, in some embodiments, an engineered nucleic acid includes a bacterial nucleotide sequence, a human nucleotide sequence, and/or a viral nucleotide sequence.

Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with naturally occurring nucleic acid molecules. Recombinant and synthetic nucleic acids also include those molecules that result from the replication of either of the foregoing. A nucleic may comprise naturally occurring nucleotides and/or non-naturally occurring nucleotides such as modified nucleotides.

In some embodiments, a nucleic acid is present in (or on) a vector. Examples of vectors include but are not limited to bacterial plasmids, phage, cosmids, phasmids, fosmids, bacterial artificial chromosomes, yeast artificial chromosomes, viruses and retroviruses (for example vaccinia, adenovirus, adeno-associated virus, lentivirus, herpes-simplex virus, Epstein-Barr virus, fowlpox virus, pseudorabies, baculovirus) and vectors derived therefrom. In some embodiments, a nucleic acid (e.g., DNA) used as a template DNA for in vitro transcription (IVT) is present in a plasmid vector.

When applied to a nucleic acid sequence, the term “isolated” denotes that the polynucleotide sequence has been removed from its natural genetic milieu and is thus free of other extraneous or unwanted coding sequences (but may include naturally occurring 5′ and 3′ untranslated regions such as promoters and terminators) and is in a form suitable for use within genetically engineered protein production systems. Such isolated molecules are those that are separated from their natural environment.

In some embodiments, a template DNA for IVT is a nucleic acid vector. A “nucleic acid vector” is a polynucleotide that carries at least one foreign or heterologous nucleic acid fragment. A nucleic acid vector may function like a “molecular carrier”, delivering fragments of nucleic acids or polynucleotides into a host cell or as a template for IVT. In some embodiments, an IVT template encodes a 5′ untranslated region, contains an open reading frame, and encodes a 3′ untranslated region and a polyA tail. The particular nucleotide sequence composition and length of an IVT template will depend on the mRNA of interest encoded by the template.

In some embodiments the nucleic acid vector according to the invention is a circular nucleic acid such as a plasmid. In other embodiments it is a linearized nucleic acid. According to some embodiments the nucleic acid vector comprises a predefined restriction site, which can be used for linearization. The linearization restriction site determines where the vector nucleic acid is opened/linearized. The restriction enzymes chosen for linearization should preferably not cut within the critical components of the vector.

A nucleic acid vector may include an insert which may be an expression cassette or open reading frame (ORF). An “open reading frame” is a continuous stretch of DNA beginning with a start codon (e.g., methionine (ATG)), and ending with a stop codon (e.g., TAA, TAG or TGA) and encodes a protein or peptide (e.g., a therapeutic protein or therapeutic peptide). In some embodiments, an expression cassette encodes an RNA including at least the following elements: a 5′ untranslated region, an open reading frame region encoding the mRNA, a 3′ untranslated region and a polyA tail. The open reading frame may encode any mRNA sequence, or portion thereof.

In some embodiments, a nucleic acid vector comprises a 5′ untranslated region (UTR). A “5′ untranslated region (UTR)” refers to a region of an mRNA that is directly upstream (i.e., 5′) from the start codon (i.e., the first codon of an mRNA transcript translated by a ribosome) that does not encode a protein or peptide. 5′ UTRs are further described herein, for example in the section entitled “Untranslated Regions”.

In some embodiments, a nucleic acid vector comprises a 3′ untranslated region (UTR). A “3′ untranslated region (UTR)” refers to a region of an mRNA that is directly downstream (i.e., 3′) from the stop codon (i.e., the codon of an mRNA transcript that signals a termination of translation) that does not encode a protein or peptide. 3′ UTRs are further described herein, for example in the section entitled “Untranslated Regions”.

The terms 5′ and 3′ are used herein to describe features of a nucleic acid sequence related to either the position of genetic elements and/or the direction of events (5′ to 3′), such as e.g., transcription by RNA polymerase or translation by the ribosome which proceeds in 5′ to 3′ direction. Synonyms are upstream (5′) and downstream (3′). Conventionally, DNA sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left-hand side, and 3′ (downstream) indicates genetic elements positioned towards the right-hand side, when following this convention.

Aspects of the disclosure relate to populations of molecules. As used herein, a “population” of molecules (e.g., DNA molecules) generally refers to a preparation (e.g., a plasmid preparation) comprising a plurality of copies of the molecule (e.g., DNA) of interest, for example a cell extract preparation comprising a plurality of expression vectors encoding a molecule of interest (e.g., a DNA encoding an RNA of interest).

A nucleic acid (e.g., mRNA) typically comprises a plurality of nucleotides. A nucleotide includes a nitrogenous base, a five-carbon sugar (ribose or deoxyribose), and at least one phosphate group. Nucleotides include nucleoside monophosphates, nucleoside diphosphates, and nucleoside triphosphates. A nucleoside monophosphate (NMP) includes a nucleobase linked to a ribose and a single phosphate; a nucleoside diphosphate (NDP) includes a nucleobase linked to a ribose and two phosphates; and a nucleoside triphosphate (NTP) includes a nucleobase linked to a ribose and three phosphates. Nucleotide analogs are compounds that have the general structure of a nucleotide or are structurally similar to a nucleotide. Nucleotide analogs, for example, include an analog of the nucleobase, an analog of the sugar and/or an analog of the phosphate group(s) of a nucleotide.

A nucleoside includes a nitrogenous base and a 5-carbon sugar. Thus, a nucleoside plus a phosphate group yields a nucleotide. Nucleoside analogs are compounds that have the general structure of a nucleoside or are structurally similar to a nucleoside. Nucleoside analogs, for example, include an analog of the nucleobase and/or an analog of the sugar of a nucleoside.

It should be understood that the term “nucleotide” includes naturally occurring nucleotides, synthetic nucleotides and modified nucleotides, unless indicated otherwise. Examples of naturally occurring nucleotides used for the production of RNA, e.g., in an IVT reaction, as provided herein include adenosine triphosphate (ATP), guanosine triphosphate (GTP), cytidine triphosphate (CTP), uridine triphosphate (UTP), and 5-methyluridine triphosphate (m5UTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO₄ to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir.

Modified nucleotides may include modified nucleobases. For example, an RNA transcript (e.g., mRNA transcript) of the present disclosure may include a modified nucleobase selected from pseudouridine (ψ), 1-methylpseudouridine (m1ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, an RNA transcript (e.g., mRNA transcript) includes a combination of at least two (e.g., 2, 3, 4 or more) of the foregoing modified nucleobases.

### Untranslated Regions

Untranslated regions (UTRs) are sections of a nucleic acid before a start codon (5′ UTR) and after a stop codon (3′ UTR) that are not translated. In some embodiments, a nucleic acid (e.g., a ribonucleic acid (RNA), e.g., a messenger RNA (mRNA)) of the disclosure comprising an open reading frame (ORF) encoding one or more proteins or peptides further comprises one or more UTR (e.g., a 5′ UTR or functional fragment thereof, a 3′ UTR or functional fragment thereof, or a combination thereof).

A UTR can be homologous or heterologous to the coding region in a nucleic acid. In some embodiments, the UTR is homologous to the ORF encoding the one or more peptide epitopes. In some embodiments, the UTR is heterologous to the ORF encoding the one or more peptide epitopes. In some embodiments, the nucleic acid comprises two or more 5′ UTRs or functional fragments thereof, each of which have the same or different nucleotide sequences. In some embodiments, the nucleic acid comprises two or more 3′ UTRs or functional fragments thereof, each of which have the same or different nucleotide sequences.

In some embodiments, the 5′ UTR or functional fragment thereof, 3′ UTR or functional fragment thereof, or any combination thereof is sequence optimized.

In some embodiments, the 5′ UTR or functional fragment thereof, 3′ UTR or functional fragment thereof, or any combination thereof comprises at least one chemically modified nucleobase, e.g., 5-methoxyuracil.

UTRs can have features that provide a regulatory role, e.g., increased or decreased stability, localization, and/or translation efficiency. A nucleic acid comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′ UTRs bear features that play roles in translation initiation. They harbor signatures like Kozak sequences that are commonly known to be involved in the process by which the ribosome initiates translation of many genes. 5′ UTRs also have been known to form secondary structures that are involved in elongation factor binding.

By engineering the features typically found in abundantly expressed genes of specific target organs, one can enhance the stability and protein production of a nucleic acid. For example, introduction of 5′ UTR of liver-expressed mRNA, such as albumin, serum amyloid A. Apolipoprotein A/B/E, transferrin, alpha fetoprotein, erythropoietin, or Factor VIII, can enhance expression of nucleic acids in hepatic cell lines or liver. Likewise, use of 5′ UTRs from other tissue-specific mRNA to improve expression in that tissue is possible for muscle (e.g., MyoD, Myosin, Myoglobin, Myogenin, Herculin), for endothelial cells (e.g., Tic-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin), and for lung epithelial cells (e.g., SP-A/B/C/D).

In some embodiments, UTRs are selected from a family of transcripts whose proteins share a common function, structure, feature, or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new nucleic acid. In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

International Patent Application No. PCT/US2014/021522 (Publ. No. WO/2014/164253) provides a listing of exemplary UTRs that may be utilized in the nucleic acids of the present disclosure as flanking regions to an ORF. This publication is incorporated by reference herein for this purpose.

Additional exemplary UTRs that may be utilized in the nucleic acids of the present disclosure include, but are not limited to, one or more 5′ UTRs and/or 3′ UTRs derived from the nucleic acid sequence of: a globin, such as an a-or B-globin (e.g., a Xenopus, mouse, rabbit, or human globin); a strong Kozak translational initiation signal; a CYBA (e.g., human cytochrome b-245 a polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV; e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., e1F4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′ UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′ UTR containing a strong Kozak translational initiation signal; a cytochrome b-245 α polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′ UTR; a DEN 3′ UTR; a PAV barley yellow dwarf virus (BYDV-PAV) 3′ UTR; an elongation factor 1 α1 (EEF1A1) 3′ UTR; a manganese superoxide dismutase (MnSOD) 3′ UTR; a β subunit of mitochondrial H(+)-ATP synthase (β-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the nucleic acids of the disclosure. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, and sequences available at www.addgene.org, the contents of each are incorporated herein by reference in their entirety. UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the nucleic acid may comprise multiple UTRs, e.g., a double, a triple or a quadruple 5′ UTR or 3′ UTR. For example, a double UTR comprises two copies of the same UTR either in series or substantially in series. For example, a double beta-globin 3′ UTR can be used (sec, for example, US2010/0129877, the contents of which are incorporated herein by reference for this purpose).

The nucleic acids of the disclosure can comprise combinations of features. For example, the ORF can be flanked by a 5′ UTR that comprises a strong Kozak translational initiation signal and/or a 3′ UTR comprising an oligo (dT) sequence for templated addition of a polyA tail. A 5′ UTR can comprise a first nucleic acid fragment and a second nucleic acid fragment from the same and/or different UTRs (see, e.g., US2010/0293625, herein incorporated by reference in its entirety for this purpose).

Other non-UTR sequences can be used as regions or subregions within the nucleic acids of the disclosure. For example, introns or portions of intron sequences can be incorporated into the nucleic acids of the disclosure. Incorporation of intronic sequences can increase protein production as well as nucleic acid expression levels. In some embodiments, the nucleic acid of the disclosure comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the nucleic acid comprises an IRES instead of a 5′ UTR sequence. In some embodiments, the nucleic acid comprises an IRES that is located between a 5′ UTR and an open reading frame. In some embodiments, the nucleic acid comprises an ORF encoding a viral capsid sequence. In some embodiments, the nucleic acid comprises a synthetic 5′ UTR in combination with a non-synthetic 3′ UTR.

In some embodiments, the UTR can also include at least one translation enhancer nucleic acid, translation enhancer element, or translational enhancer elements (collectively, “TEE,” which refers to nucleic acid sequences that increase the amount of polypeptide or protein produced from a polynucleotide. As a non-limiting example, the TEE can include those described in US2009/0226470, incorporated herein by reference in its entirety for this purpose, and others known in the art. As a non-limiting example, the TEE can be located between the transcription promoter and the start codon. In some embodiments, the 5′ UTR comprises a TEE.

In one aspect, a TEE is a conserved element in a UTR that can promote translational activity of a nucleic acid such as, but not limited to, cap-dependent or cap-independent translation. In one non-limiting example, the TEE comprises the TEE sequence in the 5′-leader of the Gtx homeodomain protein. See, e.g., Chappell et al., PNAS. 2004. 101:9590-9594, incorporated herein by reference in its entirety for this purpose.

### Poly(A) Tails

mRNAs typically include a polyA tail. A “polyA tail” is a region of mRNA that is downstream, e.g., directly downstream (i.e., 3′), from the open reading frame and/or the 3′ UTR that contains multiple, consecutive adenosine monophosphates. A polyA tail, is thus encoded by the recycled template DNA. A polyA tail may contain 10 to 300 adenosine monophosphates. For example, a poly A tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly (A) tail functions to protect mRNA from enzymatic degradation, e.g., in the cytoplasm, and aids in transcription termination, export of the mRNA from the nucleus, and translation.

In some embodiments, the polyA tail is designed relative to the length of the overall nucleic acid or the length of a particular region of the nucleic acid. This design can be based on the length of a coding region, the length of a particular feature or region or based on the length of the ultimate product expressed from the nucleic acids.

In this context, the polyA tail can be 10, 20, 30, 40, 50, 60, 70, 80, 90, or 100% greater in length than the nucleic acid. The polyA tail can also be designed as a fraction of the nucleic acid to which it belongs. In this context, the polyA tail can be 10, 20, 30, 40, 50, 60, 70, 80, or 90% or more of the total length of the construct, a construct region or the total length of the construct minus the polyA tail. Further, engineered binding sites and conjugation of nucleic acids for PolyA-binding protein can enhance expression.

### EXAMPLES

### Example 1: Recycling pDNA Template

### Isolating Recycled Template DNA

During an in vitro transcription reaction, a sample of the IVT reaction was removed and passed through a column containing particles coated with oligo-dT to capture mRNA. mRNA was transcribed and the residual reaction product containing other components of the IVT reaction, including DNA template, a tailless RNA, a non-binding RNA, and an RNA polymerase was collected. The DNA template was then isolated from the residual reaction product using membrane chromatography. Four cycles of membrane chromatography were run using a Natrix membrane to obtain a recycled DNA template solution.

Assessing Recycled pDNA Template in New IVT Reaction

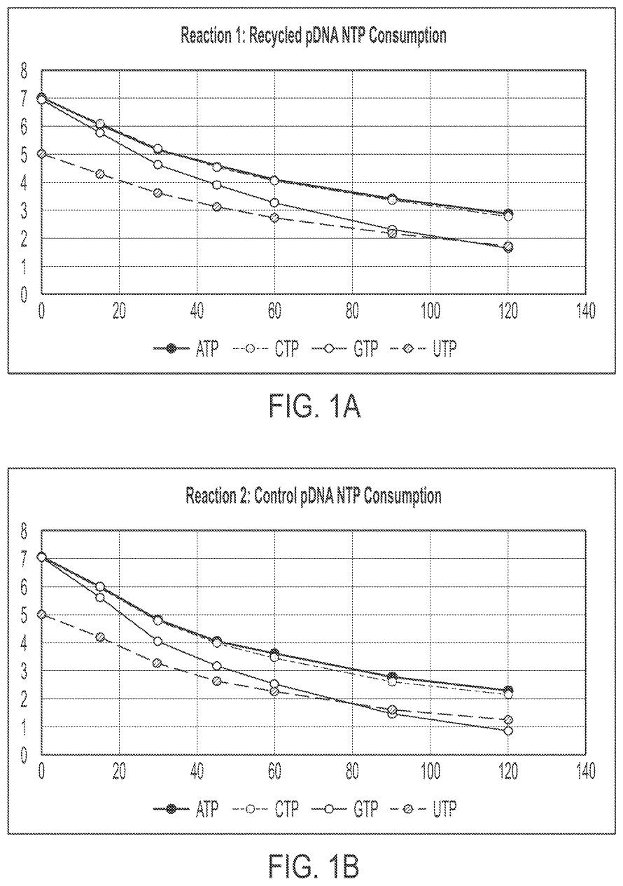

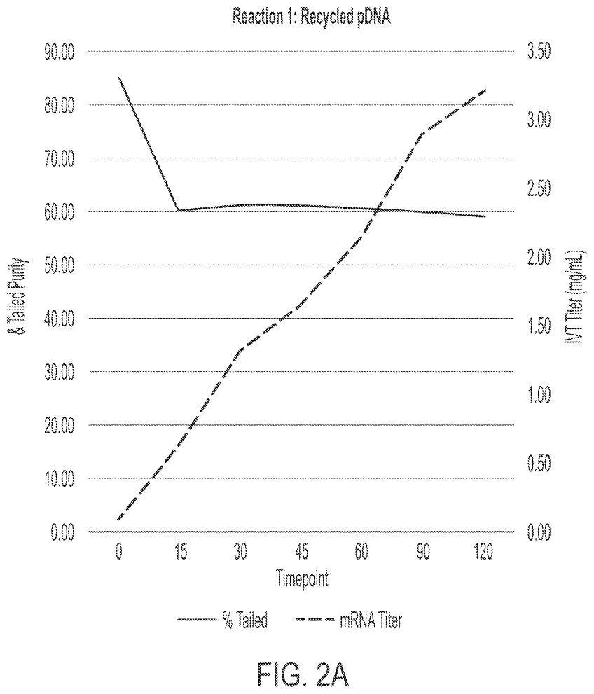

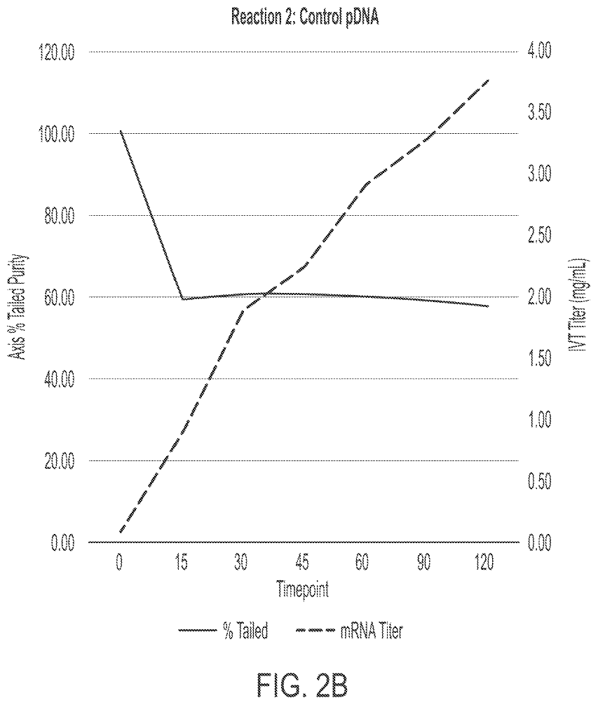

Two-IVT reactions were conducted, one using the recycled pDNA Template and the other using a control (new pDNA). During the reactions, timepoint samples were taken at 0, 15, 30, 45, 60, 90, 120 minutes and analyzed for consumption of NTP rates and purity/overall IVT productivity via NTP consumption and Tail Purity assays. NTP consumption data for recycled pDNA and control pDNA is shown in FIGS. 1A and 1B, respectively. The similarity of the NTP consumption trends indicates that both reactions are performed equivalently. Tail purity and IVT productivity was assessed and is shown for recycled pDNA and control pDNA in FIGS. 2A and 2B respectively. Similar tail purity and IVT productivity is shown for both reactions indicating that the recycled pDNA template achieves the same results as a new, non-recycled pDNA template.

### Equivalents and Scope

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or lists of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of preparing a recycled template DNA solution, the method comprising:
removing mRNA from an in vitro transcription (IVT) reaction product to obtain a solution comprising the template DNA and one or more additional IVT reaction components and/or impurities; and then
removing the one or more additional IVT reaction components and/or impurities from the solution to obtain a recycled template DNA solution.

2. The method of claim 1, wherein the one or more additional IVT reaction components comprise a RNA polymerase.

3. The method of claim 1, wherein, the one or more impurities comprise tailless mRNA and/or non-binding mRNA.

4. The method of claim 1, wherein the recycled template DNA solution is substantially free of mRNA and RNA polymerase.

5. The method of any one of claim 1-4, further comprising performing an IVT reaction using the recycled template DNA solution.

6. The method of any one of claims 1-5, wherein the one or more additional IVT reaction components and/or impurities are removed using membrane chromatography.

7. The method of claim 6, comprising performing at least four cycles of the membrane chromatography.

8. The method of any one of claims 6-7, wherein a high productivity anion exchange chromatography membrane is used in the membrane chromatography.

9. The method of any one of claims 1-8, further comprising purifying the IVT reaction product using tangential flow filtration (TFF) before and/or after removing the mRNA from the IVT reaction product.

10. The method of any one of claims 5-9, wherein the performed IVT reaction comprises combining the recycled template DNA with a RNA polymerase selected from a T7, T3 or SP6 RNA polymerase.

11. The method of claim 10, wherein the RNA polymerase is a T7 variant RNA polymerase.

12. The method of any one of claims 1-11, wherein the recycled template DNA is plasmid DNA (pDNA), PCR-amplified DNA, or complementary DNA (cDNA).

13. The method of any one of claims 1-12, wherein the recycled template DNA is 2-10 kilo base pairs (kbp) in length.

14. A system for in vitro transcription (IVT) for producing mRNA, the system, comprising
an IVT reaction chamber comprising IVT reaction components, wherein the IVT reaction components comprise at least a template DNA,
a first system for separating IVT-produced mRNA from a residual reaction product comprising the template DNA, and
a second system for separating template DNA from one or more additional IVT reaction components and/or impurities of the residual reaction product.

15. The system of claim 14, wherein the second system performs membrane chromatography.

16. The system of claim 15, wherein the membrane chromatography comprises a high productivity anion exchange chromatography membrane.

17. The system of any one of claims 14 to 16, further comprising
a third system for adding the separated template DNA back to the reaction chamber.
